(19) United States
(12) Patent Application Publication
(10) Pub. No.: US 2023/0089750 A1
(43) Pub. Date: Mar. 23, 2023

(54) Systems and Methods for Capturing a User's Movement Using Inertial Measurement Units In or On Clothing

(71) Applicant: Motusi Corporation, Portland, OR (US)

(72) Inventors: Brian Young, Portland, OR (US); Jonathan Eng, Portland, OR (US); Alexandra Peter, Portland, OR (US); Marc Alexander, Portland, OR (US)

(21) Appl. No.: 17/948,927

(22) Filed: Sep. 20, 2022

Related U.S. Application Data
(60) Provisional application No. 63/246,779, filed on Sep. 21, 2021.

(51) Int. Cl. A61B 5/00; G06F 3/0346

(52) U.S. Cl. CPC A61B 5/6804; G06F 3/0346; A61B 2562/0219

(57) ABSTRACT

The embodiments of the present disclosure are capable of capturing a user's movement using inertial measurement units (IMUs) that are embedded in, or otherwise attached to, clothing. In one embodiment, a wearable system for capturing a user's bodily movements includes a wearable item mechanically coupled to an inertial measurement unit (IMU). The wearable item, when worn by a user, positions the IMU proximate to a body part of the user. The system further includes a processor communicatively coupled to the IMU and configured to receive the sensor data from the IMU, generate movement data based on the sensor data, and transmit the movement data to a device. The device includes a processor configured to receive the movement data, generate a kinematic chain model of the user, the model representing positions and orientations of one or more joints of the user at a given time, modify the kinematic chain model based on the movement data, determine a force experienced at a joint of the user using the kinematic chain model, and record the kinematic chain model and the force over a period of time.

### CROSS REFERENCE TO RELATED APPLICATIONS

The present application claims priority to U.S. Provisional Patent Application No. 63/246,779, filed Sep. 21, 2021 and entitled “Systems and Methods for Capturing a User's Movement Using a Plurality of Inertial Measurement Units In and On Clothing,” which is incorporated by reference its entirety for all purposes.

### TECHNICAL FIELD

The present application pertains to systems and methods for capturing a user's movements using inertial measurement units (IMUs) in or on clothing, including engineered athletic wear. Additionally, the present application pertains to systems and methods for using the plurality of inertial measurement units (IMUs) embedded in clothing for dynamically determining a user's center of mass, forces and moments experienced at various parts of the body, ground reaction forces, and acceleration profiles.

### BACKGROUND

Conventionally, a person's movement is typically captured using one or more cameras (e.g., Kinect). Captured images are processed to identify the person's posture. However, such techniques for capturing a user's movement typically require the user to be in a controlled space that has, for example, one or more cameras and is well lit. Further, such techniques may require the environment behind the user to be static for acceptable performance. Accordingly, such techniques cannot be used, for example, to capture movements by a user during a live sports event in a large field, an outdoor training session, or a training session in a dark environment. Furthermore, determine the person's posture in the three-dimensional space requires a plurality of cameras positioned far apart from each other.

### SUMMARY

In one embodiment, a wearable system for capturing a user's bodily movements comprises a wearable item mechanically coupled to an inertial measurement unit (IMU), wherein the wearable item, when worn by a user, positions the IMU proximate to a body part of the user; and a processor communicatively coupled to the IMU and configured to receive the sensor data from the IMU, generate movement data based on the sensor data, and transmit the movement data to a device; wherein the device includes a processor configured to: (a) receive the movement data; (b) generate a kinematic chain model of the user, the model representing positions and orientations of one or more joints of the user at a given time, (c) modify the kinematic chain model based on the movement data, (d) determine a force experienced at a joint of the user using the kinematic chain model, and (e) record the kinematic chain model and the force over a period of time. In some embodiments, the processor of the device is further configured to initialize an orientation of the IMU by instructing the user to perform a pre-determined action. In some embodiments, the processor of the device to further configured to determine, based on the recorded model, a moment experienced at a joint of the user. In some embodiments, the processor of the device to further configured to determine, based on the recorded model, a ground reaction force. In some embodiments, the processor of the device to further configured to compare the recorded model with a previously recorded model. In some embodiments, the processor of the device to further configured to identify a region-of-interest based on an analysis of a previously recorded kinematic chain model, the region-of-interest indicating a site of an on-going injury or a site of an increased likelihood of future injury. In some embodiments, the processor of the device is further configured to replay a previously recorded model using a three-dimensional representation of a human body.

In another embodiment, a computer-readable medium storing instructions that, when executed by a computer, cause it to perform a method for capturing a user's bodily movements. The method comprises receiving movement data from a wearable system, wherein the wearable system includes: a wearable item mechanically coupled to an inertial measurement unit (IMU), wherein the wearable item, when worn by a user, positions the IMU proximate to a body part of the user; and a processor communicatively coupled to the IMU and configured to receive the sensor data from the IMU, generate the movement data based on the sensor data, and transmit the movement data; generating a kinematic chain model of the user, the model representing positions and orientations of one or more joints of the user at a given time, modifying the kinematic chain model based on the movement data, determining a force experienced at a joint of the user using the kinematic chain model, and recording the kinematic chain model and the force over a period of time. In some embodiments, the method further comprises initializing an orientation of the IMU by instructing the user to perform a pre-determined action. In some embodiments, the method further comprises determining, based on the recorded model, a moment experienced at a joint of the user. In some embodiments, the method further comprises determining, based on the recorded model, a ground reaction force. In some embodiments, the method further comprises comparing the recorded model with a previously recorded model. In some embodiments, the method further comprises identifying a region-of-interest based on an analysis of a previously recorded kinematic chain model, the region-of-interest indicating a site of an on-going injury or a site of an increased likelihood of future injury. In some embodiments, the method further comprises replaying a previously recorded model using a three-dimensional representation of a human body.

In yet another embodiment, a method for capturing a user's bodily movements comprises receiving movement data from a wearable system, wherein the wearable system includes: a wearable item mechanically coupled to an inertial measurement unit (IMU), wherein the wearable item, when worn by a user, positions the IMU proximate to a body part of the user; and a processor communicatively coupled to the IMU and configured to receive the sensor data from the IMU, generate the movement data based on the sensor data, and transmit the movement data; generating a kinematic chain model of the user, the model representing positions and orientations of one or more joints of the user at a given time, modifying the kinematic chain model based on the movement data, determining a force experienced at a joint of the user using the kinematic chain model, and recording the kinematic chain model and the force over a period of time. In some embodiments, the method further comprises initializing an orientation of the IMU by instructing the user to perform a pre-determined action. In some embodiments, the method further comprises determining, based on the recorded model, a moment experienced at a joint of the user. In some embodiments, the method further comprises determining, based on the recorded model, a ground reaction force. In some embodiments, the method further comprises comparing the recorded model with a previously recorded model. In some embodiments, the method further comprises identifying a region-of-interest based on an analysis of a previously recorded kinematic chain model, the region-of-interest indicating a site of an on-going injury or a site of an increased likelihood of future injury.

### BRIEF DESCRIPTION OF THE DRAWINGS

Embodiments of the present disclosure will be described with reference to the accompanying drawings, in which:

FIG. 1 illustrates an example of a user wearing a set of clothes in accordance with some embodiments.

FIG. 2 illustrates an example of the set of clothes in FIG. 1.

FIG. 3 illustrates an example inertial measurement unit and a portion of an example set of clothes worn by a user in accordance with some embodiments

FIG. 4 illustrates an example device for collecting sensor data from inertial measurement units, generating movement data based on the sensor data, and transmitting the movement data accordance with some embodiments.

FIG. 5 illustrates an example human kinematic chain model in accordance with some embodiments.

FIG. 6 illustrates an example system for capturing a user's movement in accordance with some embodiments.

FIG. 7 illustrates an example third-party device configured to receive feedback regarding the recorded movements and communicate the feedback to a mobile device and/or remote server, in accordance with some embodiments.

FIG. 8 illustrates an example device for identifying the recorded movement in accordance with some embodiments.

FIG. 9 illustrates movement metrics including the center of mass, on-body forces and ground reaction forces experienced by a user, in accordance with some embodiments.

FIG. 10 is a flow diagram of an example process for applying rotational vectors to a human kinematic chain model 500 to determine a new posture (i.e., 81 . . . 812) in accordance with some embodiments.

FIG. 11 is a flow diagram of a process for calculating a center of mass associated with a posture of model 500 in accordance with some embodiments.

FIG. 12 is a flow diagram of a process for calculating ground reaction forces at one or both feet in accordance with some embodiments.

FIG. 13 is a flow diagram of a process for calculating a force and moment at a connection point in a human kinematic chain model in accordance with some embodiments.

FIG. 14 is a flow diagram of a process 1200 for calculating a lumbar moment by a device in accordance with some embodiments

### DETAILED DESCRIPTION

The embodiments of the present disclosure are capable of capturing a user's movement using inertial measurement units (IMUs) that are embedded in, or otherwise attached to, clothing. In some embodiments, IMUs may be attached to regions of the clothes corresponding to arms, forearms, torso, pelvis, thigh, and leg. Based on the data from the IMUs, some embodiments are capable of dynamically determining a user's center of mass, forces and moments experienced at various parts of the body, ground reaction forces, and acceleration profiles. The movement data can be recorded and replayed (e.g., as a three-dimensional representation of a human body) and/or shared with a third party, such as a trainer or a medical professional. In some embodiments, various movement metrics, such as the center of mass, forces and moments experienced at various parts of the body, ground reaction forces, and/or acceleration profiles may be recorded along with the movements. The recorded movements and/or movement metrics may also be used for identifying irregularities in gait, injuries, suboptimal body positioning, or other problematic movements.

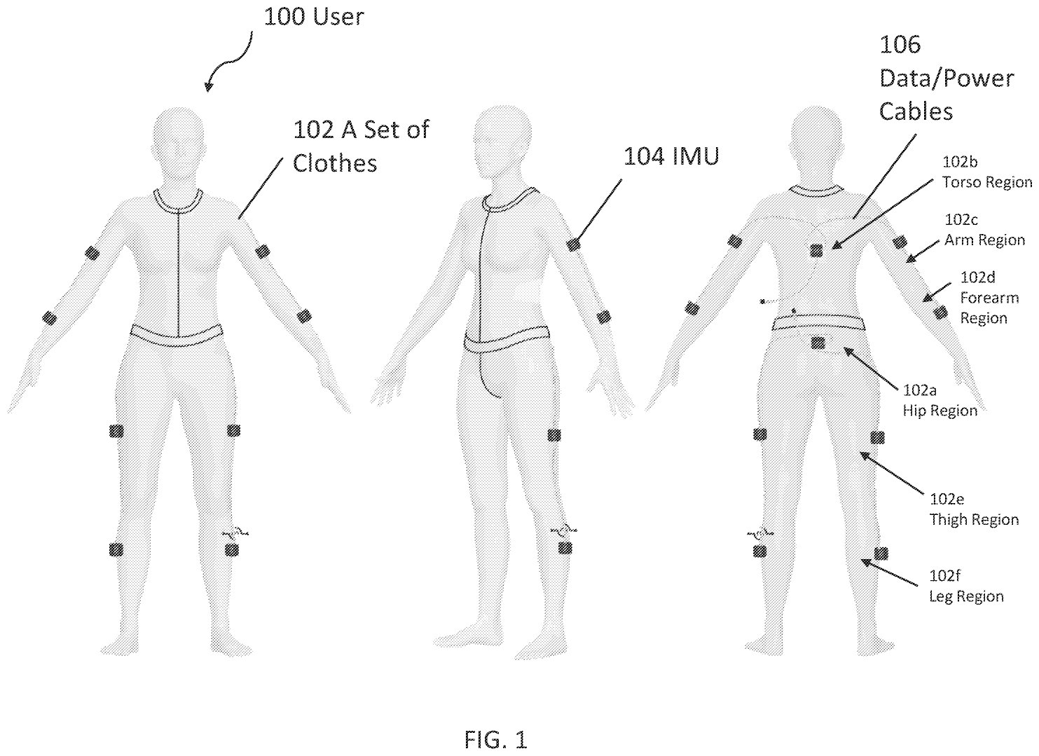

FIG. 1 illustrates an example of a user 100 wearing a set of clothes 102 in accordance with some embodiments. The set of clothes 102 includes a pelvis region 102a, a torso region 102b, two arm regions 102c, two forearm regions 102d, two thigh regions 102e, and two leg regions 102f. In some embodiments, the set of clothes 102 may include a head region (not shown), one or more feet regions (not shown), one or more hand/finger regions (not shown). As used herein, a central body region refers to either pelvis region 102a or a torso region 102. Further, as used herein, a limb region refers to either arm region 102c or a thigh region 102e.

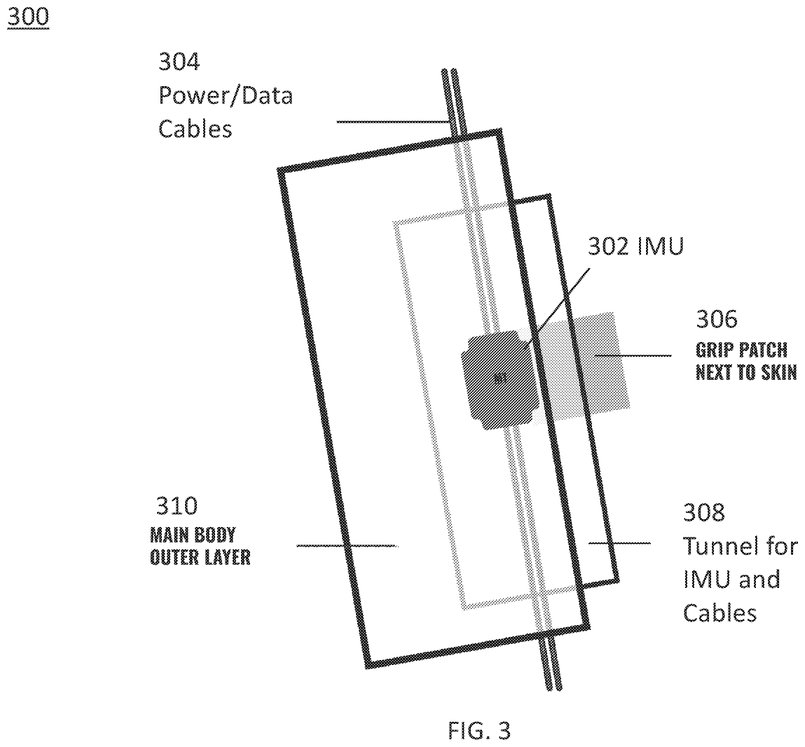

As shown in FIG. 1, the set of clothes 102 is mechanically coupled to a plurality of inertial measurement units (IMUs) 104 for capturing the user's movements. For example, IMUs 104 may be attached to the clothes 102 (e.g., outside or inside the fabric) or embedded between layers of clothes 102 as shown in FIG. 3 below.

In the example of FIG. 1, IMUs 104 are electrically connected to an on-body computer module (not shown in FIG. 1), which includes a microcontroller unit (MCU) and a battery, via one or more cables 106 that are also embedded within or run along the clothing. Cables 106 include power wires for providing power to IMUs 104. Further, Cables 106 include communication wires used to communicate sensor data from IMUs 104 to the MCU. In some embodiments, the communication wires may be a part of a series communication bus, such as an Inter-Integrated Circuit I²C bus. In these embodiments, IMUs may be connected to each other in series, and an IMU at an end of the series connection may be connected to the MCU. In some embodiments, cables 106 may be elastic cables.

In some embodiments, as shown in FIG. 1, IMUs in the top part of the clothes (e.g., torso, arm, forearm regions) may be connected together in series and, separately, IMUs in the bottom part of the clothes (e.g., pelvis, thigh, leg regions) may be connected together in series. Advantageously, separately connecting together the top IMUs and bottom IMUs allows a user to wear the top part of the clothes (e.g., shirt) without wearing the bottom part of the clothes (e.g., pants, shorts), or vice versa. For example, a user may be engaged in an activity involving only the bottom part of the body, such as cycling. Such a user may be interested only in capturing movements of the bottom part of the body. By separately connecting together the top IMUs and bottom IMUs, the user may wear only the bottom part of the clothes 102 during such an activity and capture relevant movements. In these embodiments, the IMUs in the top part and the IMUs in the bottom part may be connected to a single MCU that have two communications ports (e.g., I2C ports). Alternatively, the IMUs in the top part may be connected to a first MCU and the IMUs in the bottom part may be connected to a second MCU. Similarly, in some embodiments, the IMUs in the top part and the IMUs in the bottom part may be connected to a single battery. Alternatively, the IMUs in the top part may be connected to a first battery and the IMUs in the bottom part may be connected to a second battery. In some embodiments, all IMUs may be connected in series and connect to a single MCU and a single battery.

As shown in FIG. 1, at least one IMU is mechanically coupled to (e.g., by attaching or embedding) a central body region of the set of clothes 102. As noted above, the central body region is either a torso region or a pelvis region. Additionally, at least one IMU is mechanically coupled to a limb region of the set of clothes 102 that is directly connected to the central body region. As noted above, the limb region is either an arm region or a thigh region. In some embodiments, as shown in FIG. 1, IMUs may also be mechanically coupled to forearm regions and leg regions. IMUs may be mechanically coupled to hand/finger regions (e.g., gloves), feet regions (e.g., shoes, socks), and/or head region (e.g., hat, headband) if one or more of these regions exist in the clothes 102. IMUs in these regions may be electrically connected to the IMUs in the top and/or bottom parts of the clothes 102. Alternatively, these IMUs may be separately connected to its own MCU and/or battery, or share an MCU and/or a battery with other IMUs. For example, an IMU in the head region may be connected to the same MCU and/or battery as the top part of the clothes (e.g., IMUs in torso, arm, forearm regions) or its own MCU and/or battery. In another example, one or more IMUs in the hand/finger region(s) may be connected to the same MCU and/or battery as the top part of the clothes (e.g., IMUs in torso, arm, forearm regions) or its own MCU and/or battery. In yet another example, one or more IMUs in the feet region(s) may be connected to the same MCU and/or battery as the bottom part of the clothes (e.g., IMUs in pelvis, thigh, leg regions) or its own MCU and/or battery.

In some embodiments, a plurality of IMUs may be mechanically coupled to a single region of the set of clothes 102 to improve accuracy in capturing user's movements. In some embodiments, IMUs may be placed on the clothes 102 such that it is in the middle of two joints when worn by the user (e.g., midpoints between elbow and wrist, between elbow and shoulder, between pelvis joint and knee, between knee and ankle, between neck and pelvis, between two shoulders).

In some embodiments, one or more IMUs (e.g., four) may be mechanically coupled to back region (or pelvis/torso region) of the clothes 102.

As used herein, an IMU is a device that measures inertia experienced by the unit. In some embodiments, an IMU may include one or more accelerometers for measuring linear acceleration along a particular axis. In some embodiments, an IMU may also or instead include one or more gyroscopes for measuring angular velocity along a particular axis. In one embodiment, an IMU includes three accelerometers and three gyroscopes.

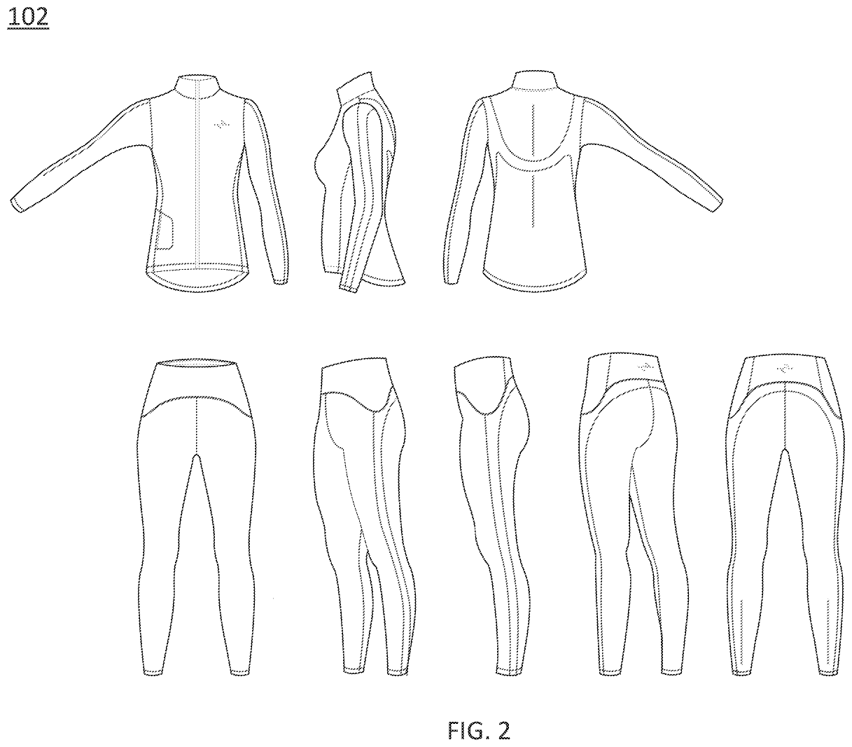

FIG. 2 illustrates an example of the set of clothes 102 in FIG. 1. As shown in FIG. 2, the set of clothes 102 includes a top (e.g., a shirt) and a bottom (e.g., pants). In some embodiments, the set of clothes may be a body suit (full or partial). In FIG. 2, the top has long sleeves. In some embodiments, however, the top may have short sleeves or no sleeves (e.g., tank top). In FIG. 2, the bottom is a pair of pants. In some embodiments, however, the bottom may be shorts. Although not shown in FIG. 2, the clothes 102 may further include headwear (e.g., beanie, headband, hat), footwear (e.g., shoes, footbed of a shoe, socks), and/or handwear (e.g., gloves). In some embodiments, clothes 102 are made of elastic material designed to fit tightly such that the IMUs move closely with the wearer's movement.

FIG. 3 illustrates an example inertial measurement unit 302 and a portion of an example set of clothes 102 worn by a user in accordance with some embodiments. As shown in FIG. 3, the set of clothes 102 has multiple layers (e.g., main body outer layer 310) which embed IMU 302. In some embodiments, one or more IMUs 302 may be attached on the outer-most layer of clothes 102. In some embodiments, the set of clothes 102 further includes a tunnel 308 (e.g., created by layers of fabric) for cables 304. In some embodiments, cables 304 may be attached on the outer-most layer of clothes 102. In some embodiments, as shown in FIG. 3, the set of clothes 102 may include a grip patch 306 under IMU 302. In some embodiments, grip patch 306 provides friction between skin and fabric of the set of clothes 102 so that IMU 302 moves closely with the wearer's body movements. In some embodiments, clothes 102 may include integrated straps for improving the mechanical coupling of the IMUs to the wearer and to minimize fit drift, for example.

In some embodiments, cables 106 may be conductive fabric that forms a layer on the clothes 102. In some embodiments, IMU 302 may be implemented as a fabric panel. In one example, one or more fabric panels may be stacked. Panels in a stack may have different type of sensors.

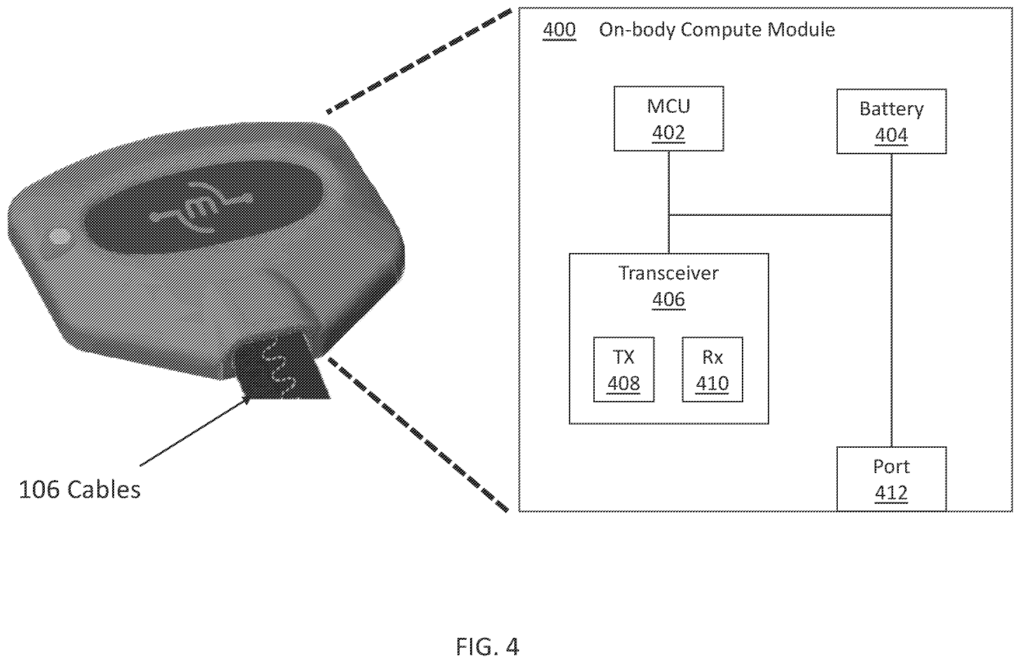

FIG. 4 illustrates an example device for collecting sensor data from inertial measurement units, generating movement data based on the sensor data, and transmitting the movement data in accordance with some embodiments. As shown in FIG. 4, the device can be an on-body computing module 400 connected to cables 106 in FIG. 1. As shown in FIG. 4, module 400 includes a microcontroller unit (MCU) 402, a battery 404, a transceiver 406, and a port 412. Port 412 is an interface for connecting to cables 106 from FIG. 1. Transceiver 406 may include a transmitter 408 and a receiver 410. Battery 404 is arranged to provide power to one or more IMUs 104 via port 412 and cables 106. In FIG. 4, module 400 is shown to be a small device that can be housed in a pocket of clothes 102. However, in some embodiments, module 400 may be a part of a smartphone. In these embodiments, cable 106 may be connected to the smartphone via the smartphone's general-purpose port (e.g., USB, Apple Lightning).

In some embodiments, MCU 402 is configured to receive sensor data from one or more IMUs 104 via port 412 and cables 106. As used herein, the sensor data is the output from the IMUs. In some embodiments, the output includes linear acceleration data from accelerometers, angular velocity data from gyroscopes, and/or orientations data derived from multiple sensing devices. In some embodiments, the output may include data generated based on the linear acceleration and/or angular velocity measured by the IMU. For example, the output may include a relative displacement data and/or a relative rotation data (i.e., the change in position and orientation). In some embodiments, the output may be provided as orientations and/or acceleration vectors. In some embodiments, MCU 402 may be configured to receive the sensor data from the IMUs at a fixed rate (e.g., at 50 Hz). In some embodiments, MCU 402 may be configured to receive sensor data at a variable rate. For example, the rate may be increased when the activity level (e.g., based on speed of movements and/or distance of moved by one or more body part) is increased and decreased when the activity level is decreased.

In the example of FIG. 4, MCU is further configured to generate movement data. As used herein, movement data refers to the data that is generated based on the sensor data received from the plurality of IMUs 104. In some embodiments, the movement data may include data generated based on the linear acceleration, angular velocity, and/or gyroscopic orientation measured by the IMU and included in the sensor data. For example, the movement data may include a relative displacement data and/or a relative rotation data (i.e., the change in position and orientation). In some embodiments, the movement data may be provided as orientations and/or acceleration vectors. In some embodiments, the movement data may include all or a portion of the sensor data. As shown in FIG. 4, MCU 402 may include a communications interface, such as an 12C interface, for receiving the sensor data from a plurality of IMUs that are connected together in series. In some embodiments, IMUs may be connected together wirelessly, for example, using Bluetooth.

As shown in FIG. 4, MCU 402 may be further configured to communicate the movement data to a remote device (not shown) via transceiver 406. The remote device may be a physical device, a virtual device, or a web service (e.g., cloud-based). In some embodiments, module 400 may include a transmitter 408 without receiver 410. In these embodiments, MCU 402 may be configured to communicate the movement data to a remote device (not shown) by broadcasting the movement data or any other one-way communication technique. In some embodiments, the transceiver 406 may be a Bluetooth transceiver and/or a Wi-Fi transceiver. In some embodiments, module 400 may include a plurality of transceivers (e.g., Bluetooth transceiver and Wi-Fi transceiver). The remote device may be, for example, a mobile device (e.g., smart watch, smartphone) or a remote server.

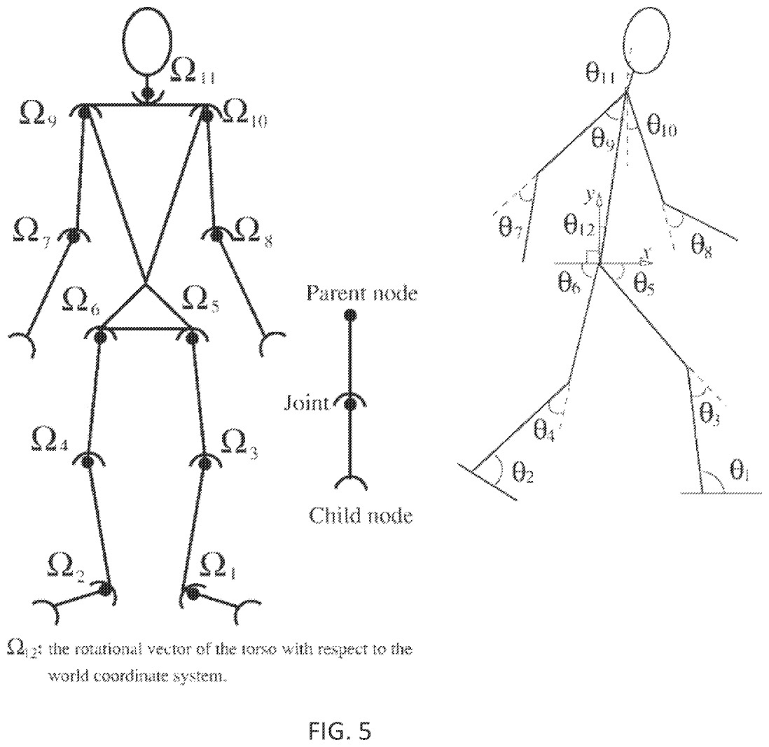

FIG. 5 illustrates an example human kinematic chain model 500 in accordance with some embodiments. In the example of FIG. 5, model 500 includes twelve independently moving body parts. By articulating the body parts through eleven joints as shown in FIG. 5 the number of degrees of freedom (DOF) is reduced significantly. Model 500 represents the current posture of a subject. As used herein, a posture is the location and orientation of body parts in model 500. A posture can be described using a twelve-dimensional vector [θ₁ . . . θ₁₂].

A body part in model 500 may have an associated length or width. In some embodiments, the length/width may be provided by a user (e.g., through the remote device). Alternatively, the length/width may be estimated based on other body measurements (e.g., waist size, jacket size) provided by a user (e.g., through the remote device). In some embodiments, the length/width may be an average length/width of a particular segment of a population (e.g., published in a scientific journal). In some embodiments, the length/width may be estimated based on an image provided by a user (e.g., through the remote device).

A body part in model 500 may be associated with a mass. In some embodiments, the mass may be provided by a user (e.g., through the remote device). Alternatively, the mass may be estimated based on other body measurements (e.g., body weight) provided by a user (e.g., through the remote device). In some embodiments, the mass may be an average mass of a particular segment of a population (e.g., published in a scientific journal). In some embodiments, the mass may be estimated based on body composition data and/or an image provided by a user (e.g., through the remote device).

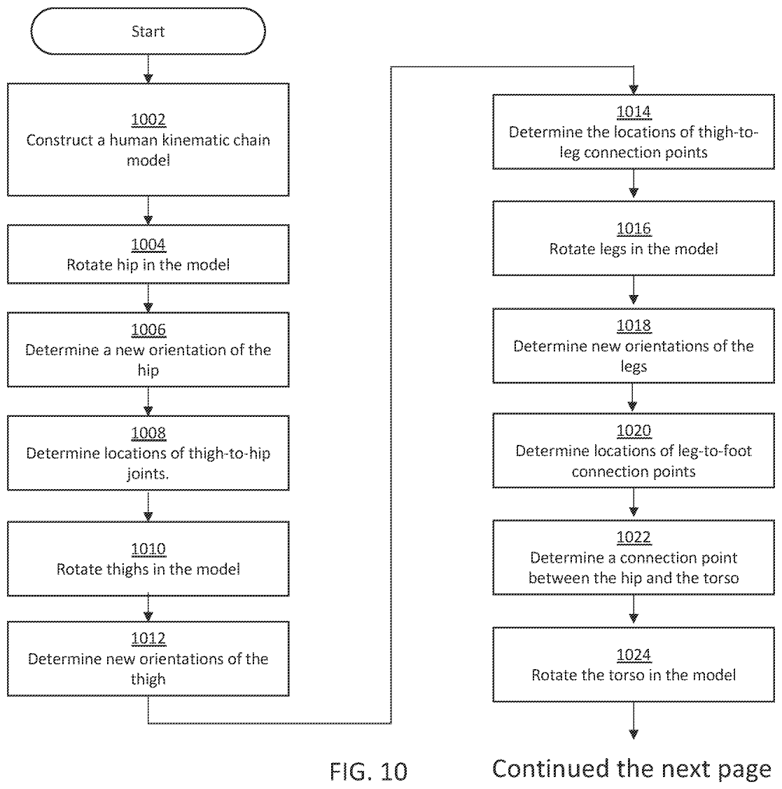

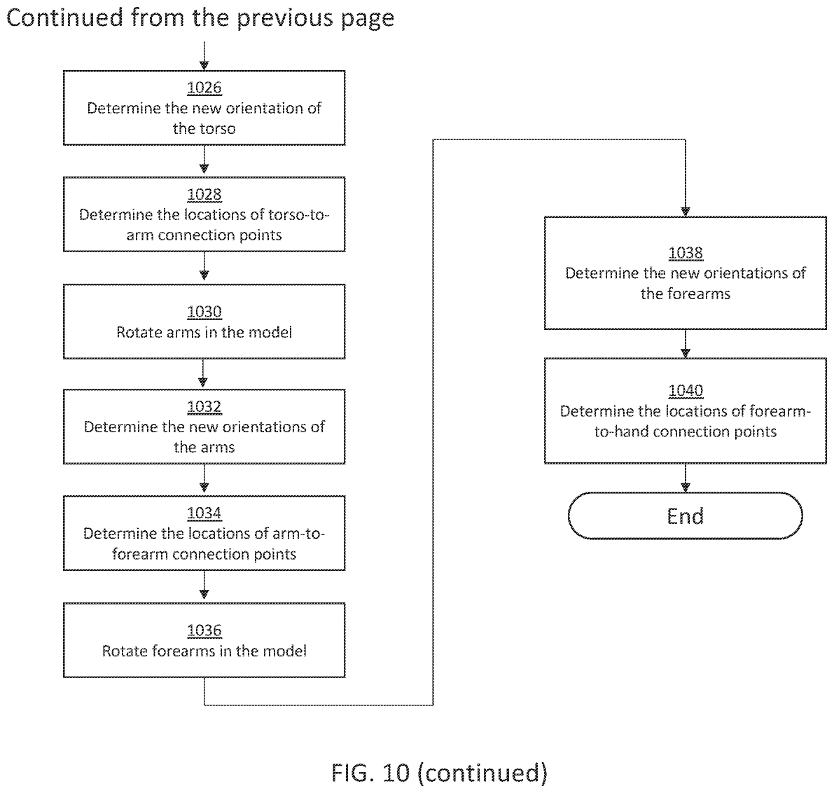

In FIG. 5, Ω is a rotational vector, which represents the amount and the direction of incremental rotation experienced at a joint. In some embodiments, rotational vectors can be calculated based on the sensor data received from the IMUs. In some embodiments, the rotational vector can be included in the sensor data. Thus, by applying the rotational vectors to human kinematic chain model 500, a new posture (i.e., θ₁ . . . θ₁₂) can be calculated. In some embodiments, a remote device constructs model 500 in its memory with a known initial posture (e.g., standing or sitting). Subsequently, the remote device may calculate a new posture using rotational vectors included in, for example, the movement data received from on-body compute module 400. In some embodiments, some or all of these calculations performed may be performed on module 400. FIG. 10 illustrates an example process for applying rotational vectors to a human kinematic chain model 500 to determine a new posture (i.e., θ₁ . . . θ₁₂).

Since the rotational vectors merely provide incremental rotation information, the remote device may initialize the posture of model 500. In some embodiments, the remote device may instruct a user to be in a predetermined posture (e.g., standing up straight) and set the posture (i.e., θ₁ . . . θ₁₂) to represent the predetermined posture. In some embodiments, the remote device may detect sensor drift or sensor movement on the body (e.g., when a body part is detected to have moved in an improbable way, such as hyperextension at a joint) based on the movement data. In these embodiments, the remote device may reinitialize the posture of model 500.

Once the model is initialized, the plurality of sensors are aligned into a common reference frame of the model 500. The alignment procedure correlates the independent sensor data to the motion in the human kinematic chain model 500. Alignment of each sensor is established using predetermined motions that the subject is instructed to perform. The alignment output is a transform that converts the independent sensor data into the subject's common reference frame. All movement data is represented in the subject's reference frame.

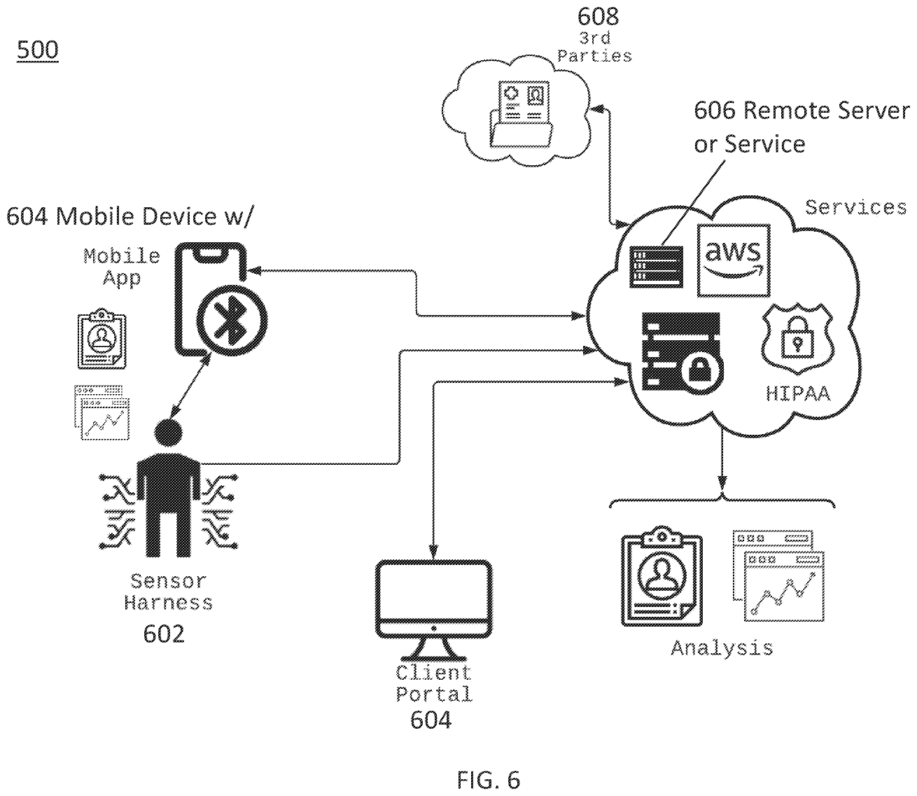

FIG. 6 illustrates an example system 600 for capturing a user's movement in accordance with some embodiments. System 600 includes a sensor harness 602, which includes clothes 102, IMUs 104, and cables 106 of FIG. 1 and one or more module(s) 400 of FIG. 4. As noted above, module 400 is configured to generate movement data based on sensor data from IMUs. Module 400 is further configured to transmit the movement data to a remote device, such as a mobile device 604 (e.g., smartphone, smart watch, tablet, laptop), a remote server 606 (implemented as a physical device, virtual device, or web service), or a third-party device 608. As used herein, a remote device refers to any device capable of receiving the movement data from Module 400 in FIG. 4. As described above, a remote device may construct a human kinematic chain model 500 and apply rotational vectors included in the movement data to determine the changes in a person's posture over time (i.e., a person's movement).

In some embodiments, the person's movement may be recorded by the remote device. For example, mobile device 604 and/or remote server 606 may record the series of the rotational vectors (e.g., as quaternions) included in the movement data or the entire movement data. Alternatively, or additionally, mobile device 604 and/or remote server 606 may record the series of resulting postures (i.e., θ₁ . . . θ₁₂) after applying the rotational vectors. The recording may further include recording of acceleration vectors, temperature, to provide some examples.

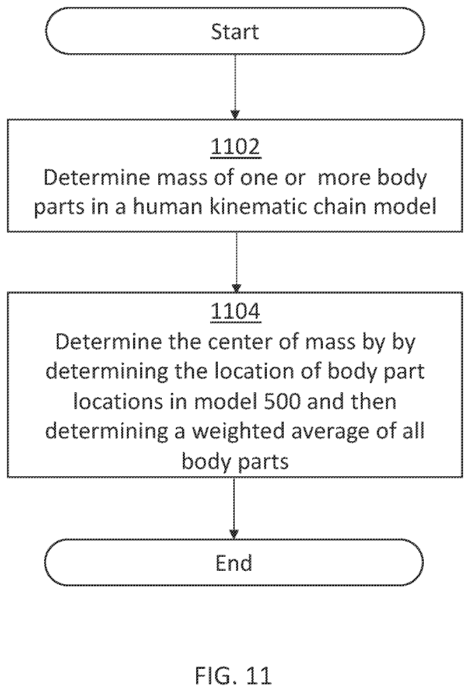

In some embodiments, the recording of a person's movement may include recording of one or more of movement metrics associated with a posture, such as the center of mass, force or moment experienced at various parts of the body, ground reaction forces, and acceleration profiles. In some embodiments, these metrics may be calculated as discussed below with respect to FIGS. 11-14. In some embodiments, an acceleration profile may be generated for any segment of the body. As used herein, an acceleration profile refers to the acceleration of a body segment over a period of time. The acceleration may be calculated or measured directly. The acceleration profile may be used to characterize a subject's performance level, fatigue, or health.

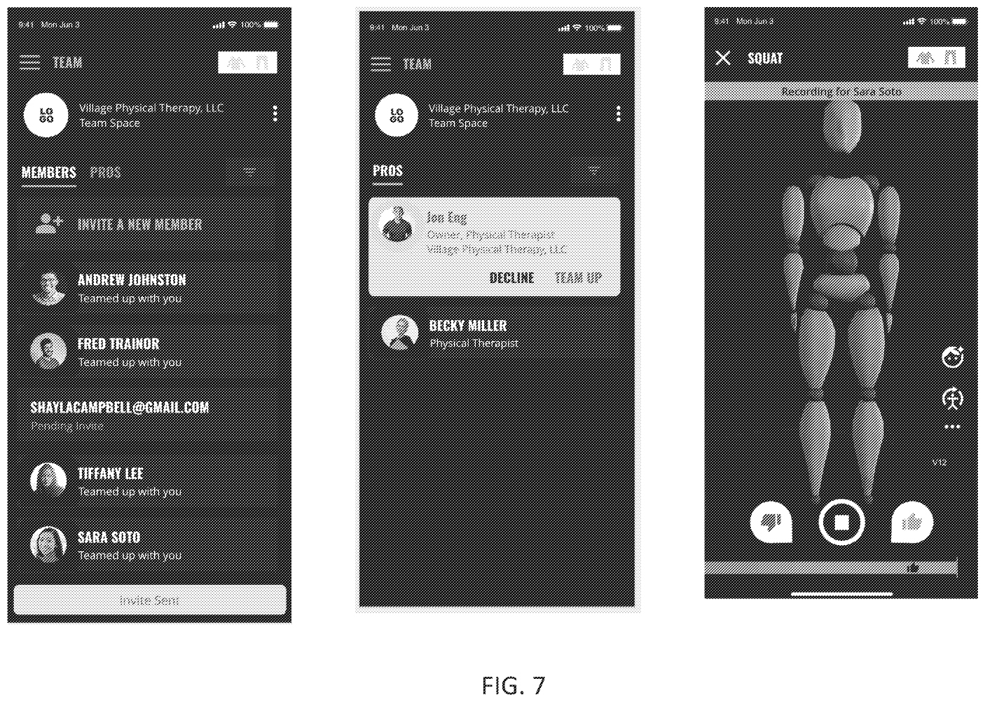

In some embodiments, a remote device may transmit the recorded movement data and metrics to another remote device (e.g., mobile device 604 to third party's device 608). The third party's device 608 may be, for example, a device operated or owned by the user's trainer or medical professional (e.g., physical therapist). In some embodiments, as shown in FIG. 7, the third party's device 608 may be configured to receive feedback regarding the recorded movements and communicate the feedback to mobile device 604 and/or remote server 606. The feedback may be text and/or multimedia data generated by the third-party device 608 or its user. Additionally, or alternatively, the feedback may be additional data or analytic data generated by the third party device 608 or its user. The feedback may be viewed by the user via mobile device 604 and/or client portal 604. In some embodiments, mobile device 604 and/or remote server 606 may stream the recorded movement to another remote device, such as a third party's device 608, such that the third party's device 608 can view a person's movement in real time. Third party's device 608 may be connected to the remote device via a local network (e.g., LAN) or via the Internet. Thus, device 608 may be in the vicinity of the user or in another city.

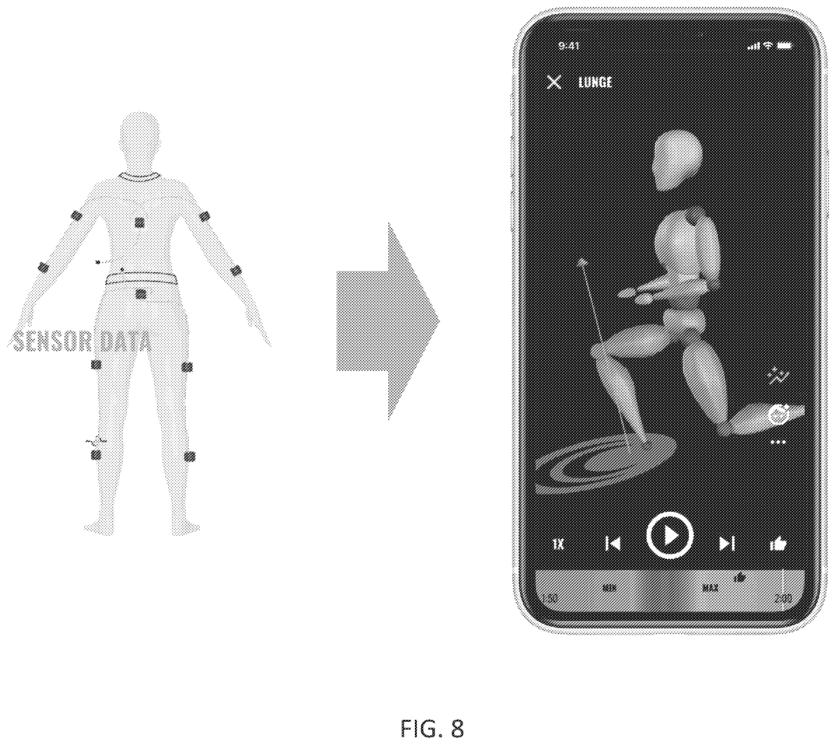

In some embodiments, the recorded movement can be replayed on a remote device. For example, the recorded movement can be replayed on mobile device 602 via an app (as shown in FIG. 8) or on a client portal 604, which can be accessed by any device (including, for example, mobile device 602 and third party device 608) with a web browser capability. As shown in FIG. 8, the recorded movements can be shown as a three-dimensional representation of a human body. In some embodiments, the three-dimensional representation may be generated using a gaming engine, such as Unity Engine.

In some embodiments, a remote device may be configured to analyze the recorded or current movement to identify a type of movement that was or is being performed by a user. In the example of FIG. 8, the recorded movement is identified as being lunge movement. In another example, the type of movement can be more specific such as “bad lunge,” “good lunge,” “perfect lunge” to provide user information about the quality of the movement performed.

In some embodiments a remote device may be configured to generate a signature associated with a recorded movement. In some embodiments, a signature may be generated based on a plurality of movements recorded and associated with a particular user. The signature may allow a system to identify the user based on the recorded movement.

In some embodiments, a remote device may be configured to compare at least two previously recorded movements or a single previously recorded movement with the current movement of the person. For example, the remote device may be configured to replay side-by-side two recorded movements or a recorded movement and the current movement for visual comparison by a user, trainer, or medical professional. Alternatively, or additionally, the movements may be replayed overlayed on top of each other for visual comparison by a user, trainer, or medical professional. In some embodiments, a remote device may perform the comparisons to identify signs of fatigue or signs of movement improvement (e.g., to determine when a user is ready to return to sports). The comparison may include comparing recorded movement metrics such as center of mass, lumbar moment, and ground reaction force. The ability to track these metrics allows the user, its trainer, or a medical professional to better understand when the user begins to experience fatigue because the pattern of the center of mass will begin to deviate away from the characteristic pattern (e.g., pattern identified from the recordings), which will then lead to changes in the magnitudes of all forces experienced on the body, including lumbar moment and ground reaction forces. Generally, an increase in anomalies in the movement data, increases the determinant of fatigue.

In some embodiments, a remote device may be configured to identify a problematic movement in the recorded movement. For example, the remote device may analyze previously recorded movements (e.g., movements of a single or multiple users) that led to an injury to identify a pattern of movements that is likely to lead to an injury. Subsequently, the remote device may determine whether the identified pattern exists in the recorded movement to predict potential injury. In some embodiments, machine learning techniques may be used to identify these patterns and/or to determine whether the identified pattern exists in the recorded movements. Advantageously, the use of machine learning techniques may identify or aid a trainer/medical professional in identifying difficult-to-detect problematic movements.

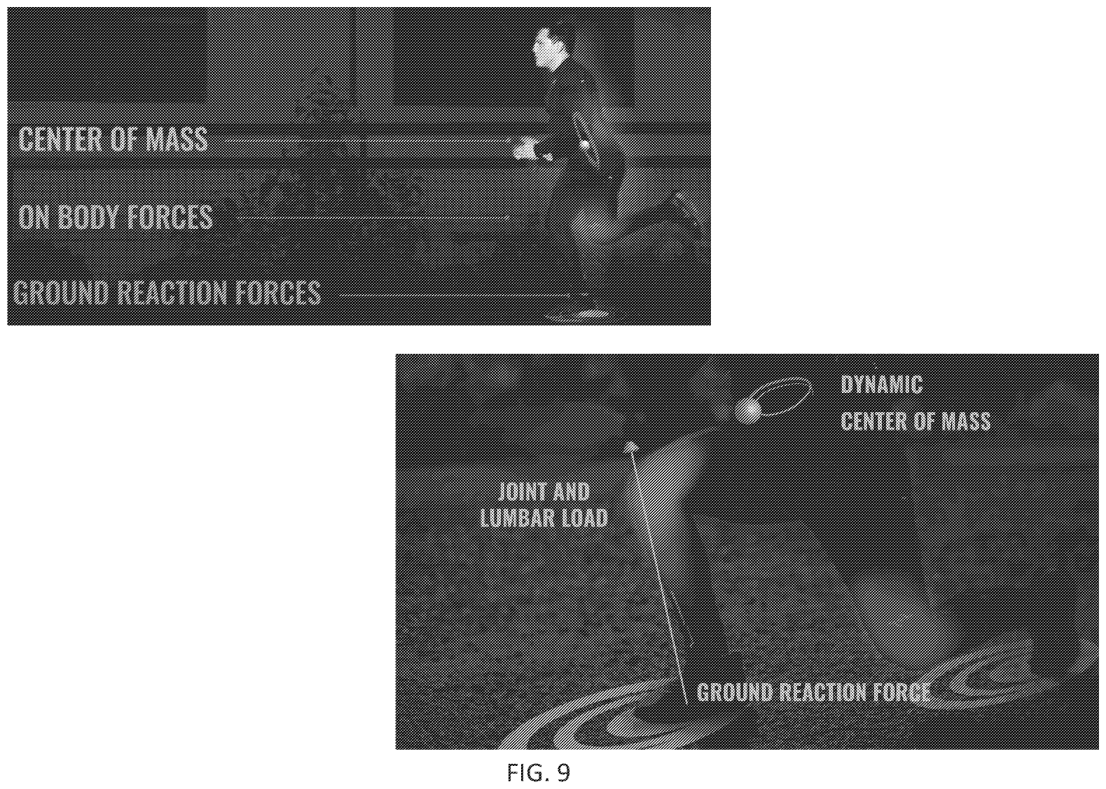

FIG. 9 illustrates movement metrics including the center of mass, forces and moments, and ground reaction forces experienced by a user. As used herein, the center of mass is the unique point at the center of a distribution of the mass of each body segment in space. Thus, the center of mass changes as one or more body parts move and changes the posture in model 500. As used herein, on-body forces refer to the amount and the direction of force experienced at a joint or any location on the body. A moment is caused by a force applied to a lever arm. This force is caused by the gravity acting on the body parts at the location of support (e.g., a joint like the knee). Additionally, the acceleration of body parts supported by the joint may further contribute to this force. Ground reaction forces refer to the reaction force at a person's foot or feet, depending on whether one or both feet are in contact with the ground. While not shown in FIG. 7, movement metrics may further include a moment for any location on the body (e.g., lumbar). A moment is the torque (i.e., force at a normal to a distance) experienced at any point (e.g., moment at the knee is the force generated by the center of mass multiplied by the distance from the knee to the center of mass in the plane normal to gravity.

FIG. 10 is a flow diagram of an example process 1000 for applying rotational vectors to a human kinematic chain model 500 to determine a new posture (i.e., θ₁ . . . θ₁₂) in accordance with some embodiments. In some embodiments, a remote device (e.g., remote device 604, remote server 606, or third party device 608) may be configured to perform process 1000.

At a step 1002, the remote device constructs a human kinematic chain model 500. In some embodiments, the remote device may further initialize model 500 by instructing a user to be in a predetermined position and setting the posture of model 500 to represent the predetermined position (e.g., sitting or standing).

At a step 1004, the remote device rotates the pelvis in model 500 by applying a rotational vector associated with pelvis. The vector may be calculated based on sensor data. Alternatively, the vector may be included in the sensor data. In some embodiments, the calculation may be performed using quaternions or any mathematical orientation representation.

At a step 1006, the remote device may determine a new orientation of the pelvis.

At a step 1008, based on the new orientation of the pelvis, the remote device may determine locations of thigh-to-pelvis joints. In some embodiments, the locations of the thing-to-pelvis joints may be determined further based on a pelvis length.

At a step 1010, the remove device may rotate thighs in model 500 by applying rotation vectors associated with respective thighs. The vectors may be calculated based on sensor data. Alternatively, the vectors may be included in the sensor data. In some embodiments, the calculation may be performed using quaternions.

At a step 1012, the remote device may determine the new orientations of the thighs.

At a step 1014, based on lengths of the thigh(s), the remote device may determine the locations of thigh-to-lower-leg connection points (i.e., knees).

At a step 1016, the remote device may rotate legs in model 500 by applying rotation vectors associated with respective legs. The vectors may be calculated based on sensor data. Alternatively, the vectors may be included in the sensor data. In some embodiments, the calculation may be performed using quaternions.

At a step 1018, the remote device may determine the new orientations of the legs.

At a step 1020, based on lengths of the leg(s), the remote device may determine the location of leg-to-foot connection points (i.e., ankles).

At an optional step, the orthogonal distance (relative to the ground) between each foot and the pelvis is calculated. A longer distance may be used to determine the location of the pelvis relative to the ground and to determine the ground reaction force distribution. In some embodiments, the orthogonal direction relative to the ground may be determined using accelerometers of one or more IMUs.

At a step 1022, the remote device may determine a connection point between the pelvis and the torso based on the new orientation of the pelvis at step 1006.

At a step 1024, the remote device may rotate the torso in model 500 by applying a rotation vector associated with the torso. The vector may be calculated based on sensor data. Alternatively, the vector may be included in the sensor data. In some embodiments, the calculation may be performed using quaternions.

At a step 1026, the remote device may determine the new orientation of the torso.

At a step 1028, based on the new orientation of the torso, lengths of the torso, and/or a width of shoulder, the remote device may determine the locations of torso-to-arm connection points (i.e., shoulders).

At a step 1030, the remote device may rotate arms in model 500 by applying rotational vectors associated with respective arms. The vectors may be calculated based on sensor data. Alternatively, the vectors may be included in the sensor data. In some embodiments, the calculation may be performed using quaternions.

At a step 1032, the remote device may determine the new orientations of the arms.

At a step 1034, based on lengths of the arm(s), the remote device may determine the locations of arm-to-forearm connection points (i.e., elbow).

At a step 1036, the remote device may rotate forearms in model 500 by applying rotational vectors associated with respective arms. The vectors may be calculated based on sensor data. Alternatively, the vectors may be included in the sensor data. In some embodiments, the calculation may be performed using quaternions.

At a step 1038, the remote device may determine the new orientations of the forearms.

At a step 1040, based on lengths of the forearm(s), the remote device may determine the locations of forearm-to-hand connection points (i.e., wrists).

In some embodiments, the process may further include additional steps of applying rotations and determining location(s) of joints to determine the new orientations of head, hand, fingers, and feet.

As noted above, a length or width of a body part may be provided by a user. Alternatively, the length/width may be estimated based on other body measurements (e.g., waist size, jacket size) provided by a user. In some embodiments, the length/width may be an average length/width measured among a particular segment (or a combination of multiple segments) of a population (e.g., published in a scientific journal). In some embodiments, the length/width may be estimated based on an image provided by a user (e.g., through the remote device).

FIG. 11 is a flow diagram of a process 1100 for calculating a center of mass associated with a posture of model 500 in accordance with some embodiments. After applying rotations and determining orientations of body parts in model 500, the center of mass associated with the new posture in model 500 may be calculated. In some embodiments, a remote device (e.g., remote device 604, remote server 606, or third party device 608) may be configured to perform process 1100.

At a step 1102, the remote device may determine mass of one or more body parts in model 500. As noted above, the mass of a body part may be provided by a user. Alternatively, or additionally, the mass of a body part may be estimated based on other body measurements (e.g., body weight) provided by a user. For example, a certain percentage of a user's body weight (e.g., determined from a scientific literature) may be determined as the mass of the body part. In some embodiments, the mass of a body part may be based on an average mass of a particular segment of a population (e.g., published in a scientific journal). In some embodiments, the mass of a body part may be estimated based on body composition data or an image provided by a user (e.g., through the remote device).

At a step 1104, the remote device may determine the center of mass by determining the location of body part locations in model 500 and determining a weighted average of all body parts.

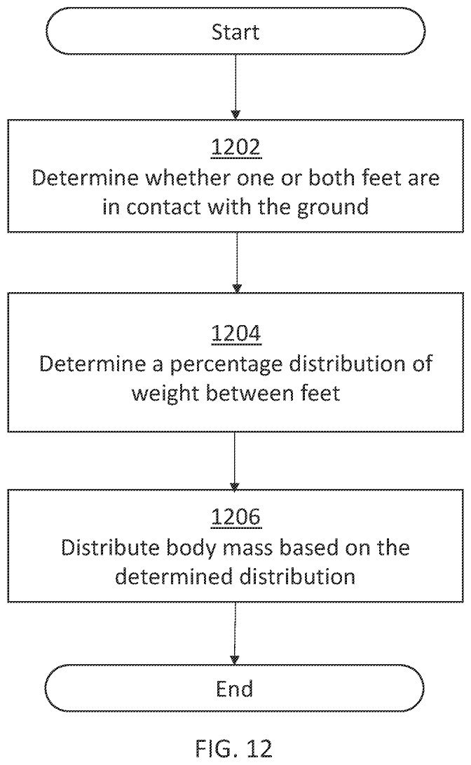

FIG. 12 is a flow diagram of a process 1200 for calculating ground reaction forces at one or both feet in accordance with some embodiments. In some embodiments, a remote device (e.g., remote device 604, remote server 606, or third party device 608) may be configured to perform process 1200.

At a step 1202, the remote device may determine whether one or both feet are in contact with the ground. In some embodiments, this may be determined by comparing the orthogonal distance (relative to the ground) between each foot and the pelvis in model 500. If the distances between both feet and the pelvis are similar, then both feet may be determined to be in contact with the ground. If one distance is larger, then the foot associated with the larger distance may be determined to be in contact with the ground.

At a step 1204, if both feet are in contact with the ground, the remote device may determine a percentage distribution of weight between feet based on the location of the center of mass relative to the location of the feet. In some embodiments, the percentage distribution is determined based on the relative location of the center of mass with respect to the feet as projected onto the ground plane.

At a step 1206, the body mass is distributed between the two feet based on the determination of which feet are in contact with the ground and/or the percentage distribution of weight between the feet. If one foot is in contact with the ground, then the reaction force is the body mass multiplied by the gravity and any acceleration experienced by the body.

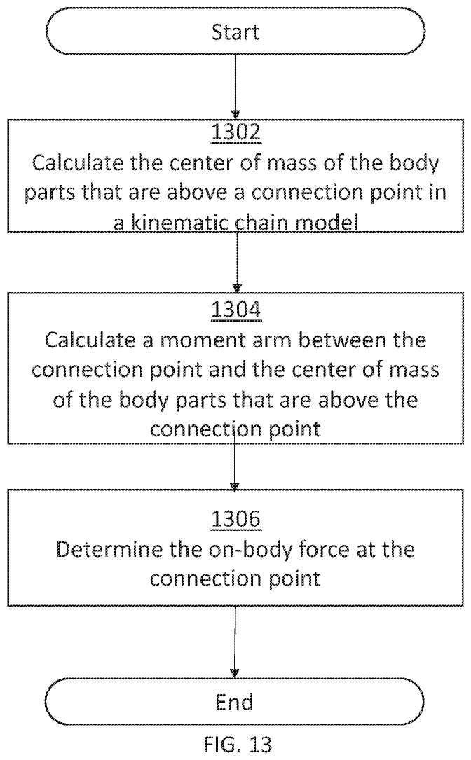

FIG. 13 is a flow diagram of a process 1300 for calculating a force and moment at a connection point in a human kinematic chain model 500 in accordance with some embodiments. In some embodiments, a remote device (e.g., remote device 604, remote server 606, or third-party device 608) may be configured to perform process 1200.

At a step 1302, the remote device may calculate a center of mass of the body parts that are above the connection point in model 500.

At a step 1304, the remote device may calculate a moment arm between the connection point and the center of mass of the body parts that are above the connection point in model 500.

At a step 1306, the remote device may determine the on-body force at the connection point by multiplying the mass of the body parts that are above the connection point with the moment arm and dividing by a distance to the force on the body. In some embodiments, the distance to the force on the body may be half of the radius of the body part associated with the connection point. For example, if the connection point is at the pelvis, the distance to the force on the body may be half of the radius of the torso. If the connection point is at a knee, the distance to the force on the body may be half of the radius of the leg.

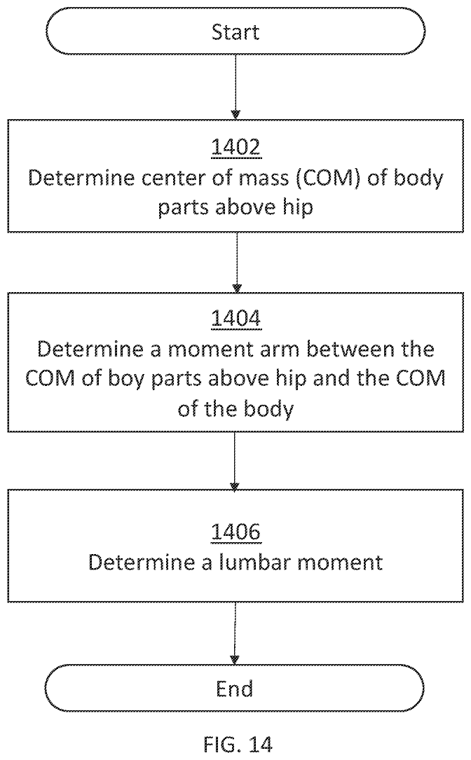

FIG. 14 is a flow diagram of a process 1200 for calculating a lumbar moment by a device in accordance with some embodiments. In some embodiments, a remote device (e.g., remote device 604, remote server 606, or third party device 608) may be configured to perform process 1200.

At a step 1402, the remote device may determine the center of mass of the body parts that are above the pelvis in model 500. In some embodiments, the body parts that are above the pelvis in model 500 includes the torso, arms and forearms. In some embodiments, the body parts that are above the pelvis in model 500 may further include hands and the head.

At a step 1404, the remote device may determine a moment arm between the center of mass of model 500 calculated in process 900 and the center of mass of model of the body parts that are above the pelvis in model 500.

At a step 1406, the remote device may determine a lumbar moment by multiplying the moment arm by (1) the mass of body parts that are above the pelvis in model 500 and (2) an amount of acceleration experienced (e.g., gravity and movement of body parts) by the body parts above the pelvis in model 500.

It will be appreciated by those skilled in the art that changes could be made to the embodiments described above without departing from the broad inventive concept thereof. It is understood, therefore, that the invention disclosed herein is not limited to the particular embodiments disclosed, and is intended to cover modifications within the spirit and scope of the present invention.

This written description describes exemplary embodiments of the invention, but other variations fall within scope of the disclosure. For example, the systems and methods may include and utilize data signals conveyed via networks (e.g., local area network, wide area network, internet, combinations thereof, etc.), fiber optic medium, carrier waves, wireless networks, etc. for communication with one or more data processing devices. The data signals can carry any or all of the data disclosed herein that is provided to or from a device.

The methods and systems described herein may be implemented on many different types of processing devices by program code comprising program instructions that are executable by the device processing system. The software program instructions may include source code, object code, machine code, or any other stored data that is operable to cause a processing system to perform the methods and operations described herein. Any suitable computer languages may be used such as C, C++, Java, etc., as will be appreciated by those skilled in the art. Other implementations may also be used, however, such as firmware or even appropriately designed hardware configured to carry out the methods and systems described herein.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, Flash memory, flat files, databases, programming data structures, programming variables, IF-THEN (or similar type) statement constructs, etc.). It is noted that data structures describe formats for use in organizing and storing data in databases, programs, memory, or other non-transitory computer-readable media for use by a computer program.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. In particular embodiments, a non-transitory computer- or machine-readable medium may be encoded with instructions in the form of machine instructions, hypertext markup language based instructions, or other applicable instructions to cause one or more data processors to perform operations. As used herein, the term “machine-readable medium” (or “computer-readable medium”) refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Finally, as used in the description herein and throughout the claims that follow, the meanings of “and” and “or” include both the conjunctive and disjunctive and may be used interchangeably unless the context expressly dictates otherwise; the phrase “exclusive or” may be used to indicate situation where only the disjunctive meaning may apply.

In the descriptions above and in the claims, phrases such as “at least one of” or “one or more of” may occur followed by a conjunctive list of elements or features. The term “and/or” may also occur in a list of two or more elements or features. Unless otherwise Implicitly or Explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in methods, systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A wearable system for capturing a user's bodily movements, comprising:
a wearable item mechanically coupled to an inertial measurement unit (IMU), wherein the wearable item, when worn by a user, positions the IMU proximate to a body part of the user; and
a processor communicatively coupled to the IMU and configured to receive the sensor data from the IMU, generate movement data based on the sensor data, and transmit the movement data to a device;
wherein the device includes a processor configured to:
(a) receive the movement data;
(b) generate a kinematic chain model of the user, the model representing positions and orientations of one or more joints of the user at a given time,
(c) modify the kinematic chain model based on the movement data,
(d) determine a force experienced at a joint of the user using the kinematic chain model, and
(e) record the kinematic chain model and the force over a period of time.

2. The system of claim 1, wherein the processor of the device is further configured to initialize an orientation of the IMU by instructing the user to perform a pre-determined action.

3. The system of claim 1, wherein the processor of the device to further configured to determine, based on the recorded model, a moment experienced at a joint of the user.

4. The system of claim 1, wherein the processor of the device to further configured to determine, based on the recorded model, a ground reaction force.

5. The system of claim 1, wherein the processor of the device to further configured to compare the recorded model with a previously recorded model.

6. The system of claim 1, wherein the processor of the device to further configured to identify a region-of-interest based on an analysis of a previously recorded kinematic chain model, the region-of-interest indicating a site of an on-going injury or a site of an increased likelihood of future injury.

7. The system of claim 1, wherein the processor of the device is further configured to replay a previously recorded model using a three-dimensional representation of a human body.

8. A computer-readable medium storing instructions that, when executed by a computer, cause it to perform a method for capturing a user's bodily movements, the method comprising:
receiving movement data from a wearable system, wherein the wearable system includes:
a wearable item mechanically coupled to an inertial measurement unit (IMU), wherein the wearable item, when worn by a user, positions the IMU proximate to a body part of the user; and
a processor communicatively coupled to the IMU and configured to receive the sensor data from the IMU, generate the movement data based on the sensor data, and transmit the movement data;
generating a kinematic chain model of the user, the model representing positions and orientations of one or more joints of the user at a given time,
modifying the kinematic chain model based on the movement data,
determining a force experienced at a joint of the user using the kinematic chain model, and
recording the kinematic chain model and the force over a period of time.

9. The computer-readable medium of claim 8, wherein the method further comprises initializing an orientation of the IMU by instructing the user to perform a pre-determined action.

10. The computer-readable medium of claim 8, wherein the method further comprises determining, based on the recorded model, a moment experienced at a joint of the user.

11. The computer-readable medium of claim 8, wherein the method further comprises determining, based on the recorded model, a ground reaction force.

12. The computer-readable medium of claim 8, wherein the method further comprises comparing the recorded model with a previously recorded model.

13. The computer-readable medium of claim 8, wherein the method further comprises identifying a region-of-interest based on an analysis of a previously recorded kinematic chain model, the region-of-interest indicating a site of an on-going injury or a site of an increased likelihood of future injury.

14. The computer-readable medium of claim 8, wherein the method further comprises replaying a previously recorded model using a three-dimensional representation of a human body.

15. A method for capturing a user's bodily movements, the method comprising:
receiving movement data from a wearable system, wherein the wearable system includes:
a wearable item mechanically coupled to an inertial measurement unit (IMU), wherein the wearable item, when worn by a user, positions the IMU proximate to a body part of the user; and
a processor communicatively coupled to the IMU and configured to receive the sensor data from the IMU, generate the movement data based on the sensor data, and transmit the movement data;
generating a kinematic chain model of the user, the model representing positions and orientations of one or more joints of the user at a given time,
modifying the kinematic chain model based on the movement data,
determining a force experienced at a joint of the user using the kinematic chain model, and
recording the kinematic chain model and the force over a period of time.

16. The method of claim 15, further comprising initializing an orientation of the IMU by instructing the user to perform a pre-determined action.

17. The method of claim 15, further comprising determining, based on the recorded model, a moment experienced at a joint of the user.

18. The method of claim 15, further comprising determining, based on the recorded model, a ground reaction force.

19. The method of claim 15, further comprising comparing the recorded model with a previously recorded model.

20. The method of claim 15, further comprising identifying a region-of-interest based on an analysis of a previously recorded kinematic chain model, the region-of-interest indicating a site of an on-going injury or a site of an increased likelihood of future injury.
